(19) United States
(12) Patent Application Publication
(10) Pub. No.: US 2013/0149731 A1
(43) Pub. Date: Jun. 13, 2013

(54) APPARATUS FOR CULTURING CELL AND ANALYZING CELL REACTION, AND METHOD OF ANALYZING CELL REACTION USING THE SAME

(71) Applicants: Jeong Won PARK, Daejeon (KR); Seungkyoung YANG, Daejeon (KR); Moon Youn JUNG, Daejeon (KR); Kibong SONG, Daejeon (KR)

(72) Inventors: Jeong Won PARK, Daejeon (KR); Seungkyoung YANG, Daejeon (KR); Moon Youn JUNG, Daejeon (KR); Kibong SONG, Daejeon (KR)

(73) Assignee: Electronics and Telecommunications Research Institute, Daejeon (KR)

(21) Appl. No.: 13/531,383

(22) Filed: Jun. 22, 2012

(30) Foreign Application Priority Data
Dec. 12, 2011 (KR) 10-2011-0133014

(51) Int. Cl. C12M 1/00; C12Q 1/02

(52) U.S. Cl. 435/29; 435/289.1

(57) ABSTRACT

Provided is an apparatus for culturing a cell and analyzing a cell reaction. The apparatus may include a plurality of cell culturing rooms, cell injection ports connected to the cell culturing rooms through cell injection channels, respectively, and used for injecting different cells from each other into the cell culturing rooms, the number of the cell injection ports being the same as that of the cell culturing rooms, and a reagent injection port to inject a reagent into the cell culturing rooms.

### CROSS-REFERENCE TO RELATED APPLICATIONS

This U.S. non-provisional patent application claims priority under 35 U.S.C. §119 to Korean Patent Application No. 10-2011-0133014, filed on Dec. 12, 2011, in the Korean Intellectual Property Office, the entire contents of which are hereby incorporated by reference.

### BACKGROUND OF THE INVENTION

Embodiments of the inventive concepts relate to a cell culturing apparatus, and in particular, an apparatus for culturing different cells from each other and analyzing cell reactions caused by a stimulating material, and a cell-reaction analyzing method using the same.

A cell culturing is an important technology in the field of biological, medical and life sciences. For the last ten years, the main consideration to the cell culturing technology is to provide a high-efficiency cell-based assays aiming at developing a new drug and cell biology. For example, a high-efficiency cell-based screening platforms have been developed due to the need for the pharmaceutical industry, and cell-based drug-screening assays are being used to study interaction between various molecules. Analysis apparatus has been developed and used in a form of a micro-well plate, but it suffers from technical problems, such as high operating cost and insufficiency of measurable period.

To overcome these problems, cell assays using a micro-fluid device have been developed. Although a highly efficient cell-based assaying apparatus have been developed to realize a long-term cell culturing, due to its structural reason, they have been used in measuring only one species of cell and in a structure capable of forming concentration gradient on one species of drug. Alternatively, there has been developed single-cell-array type apparatus, in which cells of the same kind spaced apart from each other are arranged using a plurality of traps or wells. However, this apparatus has a limitation that acquirable information is limited to an effect of one drug on one species of cell, despite of its single cell array.

Recently, there has been developed an apparatus, in which cells of various kinds obtained from collenchyma are randomly arranged in wells. This can be used to analyze a reaction pattern of odor molecules, but has a technical problem, such as poor reproducibility in reaction pattern.

### SUMMARY

Embodiments of the inventive concepts provide an apparatus, in which cells of various species can be arranged and cultured at predetermined positions and provide a reaction pattern with reproducibility with respect to a specific stimulating material.

Other embodiments of the inventive concepts provide a method of analyzing cell reactions using the apparatus.

According to example embodiments of the inventive concepts, an apparatus for culturing and analyzing cells may include a plurality of cell culturing rooms, cell injection ports connected to the cell culturing rooms through cell injection channels, respectively, and used for injecting different cells from each other into the cell culturing rooms, the number of the cell injection ports being the same as that of the cell culturing rooms, and a reagent injection port to inject a reagent into the cell culturing rooms.

In example embodiments, the cell culturing rooms may be connected, in parallel or in series, to the reagent injection port by at least one reagent injection channel.

In example embodiments, the cell culturing rooms may be arranged in the form of a matrix. For example, the reagent injection channel may include a plurality of channels branching off from the reagent injection port, and ones of the cell culturing rooms in each row of the matrix arrangement may be connected in series to the corresponding one of the channels. The apparatus may further include at least one cell-containing solution exhaust channel, each of which is connected in common to ones of the cell culturing rooms in each row of the matrix arrangement. The apparatus may further include a cell-containing solution outlet connected to the cell-containing solution exhaust channel to exhaust the cell-containing solution. The cell culturing rooms may be arranged to have the form of an M×N matrix. A total area of the cell culturing rooms may be configured in such a way that the total area can be completely seen by a microscope with 40× magnification.

In example embodiments, the reagent may include a cell culturing solution or a stimulating material.

In example embodiments, each of the cell culturing rooms may include a cell capturing structure with a plurality of pillars arranged along an edge of the cell culturing room, from a plan view.

In example embodiments, the apparatus may further include a reagent outlet connected to portions of the cell culturing rooms opposite the reagent injection port to exhaust the reagent.

According to example embodiments of the inventive concepts, a method of analyzing a cell reaction may include injecting different cells from each other into a plurality of cell culturing rooms, injecting a culturing solution into the cell culturing rooms to culture the cells, and injecting a stimulating material into all of the cell culturing rooms. Cell reactions of the cells caused by the stimulating material may be analyzed by examining visualized patterns occurring in the cell culturing rooms.

In example embodiments, the cell culturing rooms may be arranged to have the form of an M×N matrix. A total area of the cell culturing rooms may be configured in such a way that the total area can be completely seen by a microscope with 40× magnification.

### BRIEF DESCRIPTION OF THE DRAWINGS

Example embodiments will be more clearly understood from the following brief description taken in conjunction with the accompanying drawings. The accompanying drawings represent non-limiting, example embodiments as described herein.

FIG. 1 is a schematic plan view illustrating cell reaction patterns obtained by using an apparatus for culturing a cell and analyzing a cell reaction according to example embodiments of the inventive concept, and in particular, by applying different stimulating materials to 32 groups of cells;

FIG. 2A is a plan view illustrating an apparatus for culturing a cell and analyzing a cell reaction according to example embodiments of the inventive concept and a method of analyzing a cell reaction with the same, and FIG. 2B is an enlarged plan view of a portion of FIG. 2A;

FIGS. 3A and 3B are plan views illustrating results of cell culturing obtained by using an apparatus for culturing a cell and analyzing a cell reaction according to example embodiments of the inventive concept, and in particular, obtained from two different cells arranged at desired culturing rooms.

It should be noted that these figures are intended to illustrate the general characteristics of methods, structure and/or materials utilized in certain example embodiments and to supplement the written description provided below. These drawings are not, however, to scale and may not precisely reflect the precise structural or performance characteristics of any given embodiment, and should not be interpreted as defining or limiting the range of values or properties encompassed by example embodiments. For example, the relative thicknesses and positioning of molecules, layers, regions and/or structural elements may be reduced or exaggerated for clarity. The use of similar or identical reference numbers in the various drawings is intended to indicate the presence of a similar or identical element or feature.

### DETAILED DESCRIPTION

Example embodiments of the inventive concepts will now be described more fully with reference to the accompanying drawings, in which example embodiments are shown. Example embodiments of the inventive concepts may, however, be embodied in many different forms and should not be construed as being limited to the embodiments set forth herein; rather, these embodiments are provided so that this disclosure will be thorough and complete, and will fully convey the concept of example embodiments to those of ordinary skill in the art. In the drawings, the thicknesses of layers and regions are exaggerated for clarity. Like reference numerals in the drawings denote like elements, and thus their description will be omitted.

It will be understood that when an element is referred to as being “connected” or “coupled” to another element, it can be directly connected or coupled to the other element or intervening elements may be present. In contrast, when an element is referred to as being “directly connected” or “directly coupled” to another element, there are no intervening elements present. Like numbers indicate like elements throughout. As used herein the term “and/or” includes any and all combinations of one or more of the associated listed items. Other words used to describe the relationship between elements or layers should be interpreted in a like fashion (e.g., “between” versus “directly between,” “adjacent” versus “directly adjacent,” “on” versus “directly on”).

It will be understood that, although the terms “first”, “second”, etc. may be used herein to describe various elements, components, regions, layers and/or sections, these elements, components, regions, layers and/or sections should not be limited by these terms. These terms are only used to distinguish one element, component, region, layer or section from another element, component, region, layer or section. Thus, a first element, component, region, layer or section discussed below could be termed a second element, component, region, layer or section without departing from the teachings of example embodiments.

Spatially relative terms, such as “beneath,” “below,” “lower,” “above,” “upper” and the like, may be used herein for ease of description to describe one element or feature's relationship to another element(s) or feature(s) as illustrated in the figures. It will be understood that the spatially relative terms are intended to encompass different orientations of the device in use or operation in addition to the orientation depicted in the figures. For example, if the device in the figures is turned over, elements described as “below” or “beneath” other elements or features would then be oriented “above” the other elements or features. Thus, the exemplary term “below” can encompass both an orientation of above and below. The device may be otherwise oriented (rotated 90 degrees or at other orientations) and the spatially relative descriptors used herein interpreted accordingly.

The terminology used herein is for the purpose of describing particular embodiments only and is not intended to be limiting of example embodiments. As used herein, the singular forms “a,” “an” and “the” are intended to include the plural forms as well, unless the context clearly indicates otherwise. It will be further understood that the terms “comprises” “comprising”, “includes” and/or “including,” if used herein, specify the presence of stated features, integers, steps, operations, elements and/or components, but do not preclude the presence or addition of one or more other features, integers, steps, operations, elements, components and/or groups thereof.

Example embodiments of the inventive concepts are described herein with reference to cross-sectional illustrations that are schematic illustrations of idealized embodiments (and intermediate structures) of example embodiments. As such, variations from the shapes of the illustrations as a result, for example, of manufacturing techniques and/or tolerances, are to be expected. Thus, example embodiments of the inventive concepts should not be construed as limited to the particular shapes of regions illustrated herein but are to include deviations in shapes that result, for example, from manufacturing. For example, an implanted region illustrated as a rectangle may have rounded or curved features and/or a gradient of implant concentration at its edges rather than a binary change from implanted to non-implanted region. Likewise, a buried region formed by implantation may result in some implantation in the region between the buried region and the surface through which the implantation takes place. Thus, the regions illustrated in the figures are schematic in nature and their shapes are not intended to illustrate the actual shape of a region of a device and are not intended to limit the scope of example embodiments.

Unless otherwise defined, all terms (including technical and scientific terms) used herein have the same meaning as commonly understood by one of ordinary skill in the art to which example embodiments of the inventive concepts belong. It will be further understood that terms, such as those defined in commonly-used dictionaries, should be interpreted as having a meaning that is consistent with their meaning in the context of the relevant art and will not be interpreted in an idealized or overly formal sense unless expressly so defined herein.

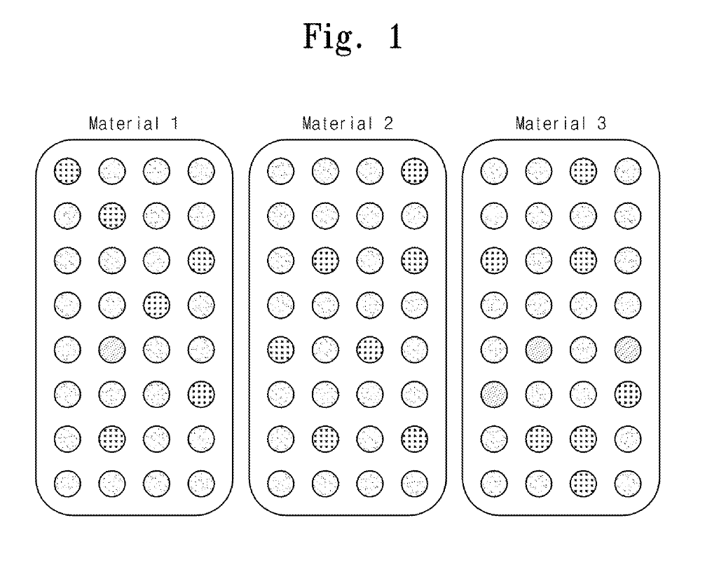

FIG. 1 is a schematic plan view illustrating cell reaction patterns obtained by using an apparatus for culturing a cell and analyzing a cell reaction according to example embodiments of the inventive concept, and in particular, by applying different stimulating materials to 32 groups of cells.

FIG. 1 shows cell reaction patterns of 32 cells obtained by using stimulating materials (indicated by “Material 1”, “Material 2”, and “Material 3”) different from each other. In FIG. 1, the darker the shade of the pattern means the greater an intensity of the cell reaction that can be observed. The use of different stimulating materials from each other resulted in a difference between the cell reaction patterns of 32 cells. Since positions of the 32 cells were given by a designer, it is possible to infer an unknown stimulating material from the cell reaction patterns.

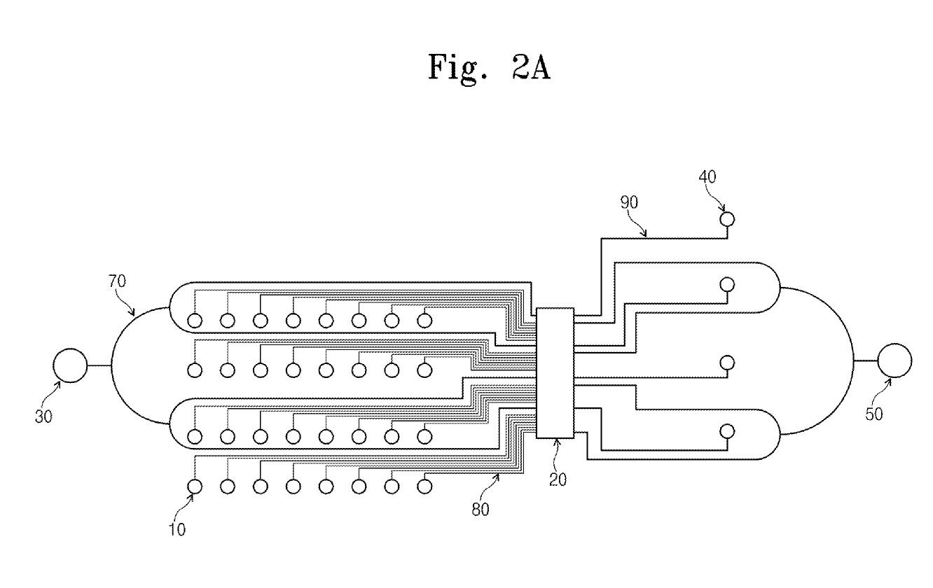

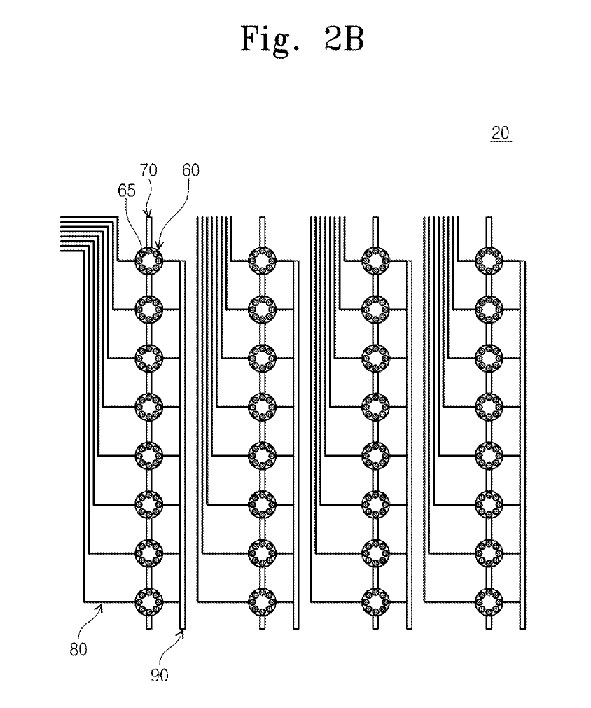

FIG. 2A is a plan view illustrating an apparatus for culturing a cell and analyzing a cell reaction according to example embodiments of the inventive concept and a method of analyzing a cell reaction with the same, and FIG. 2B is an enlarged plan view of a portion of FIG. 2A.

Referring to FIGS. 2A and 2B, an apparatus for culturing and analyzing cells may include a cell culturing array 20 with a plurality of cell culturing rooms 60, cell injection ports 10 connected to the cell culturing rooms 60 through cell injection channels 80, respectively, and a reagent injection port 30 to be used to inject a reagent into the cell culturing rooms 60. In example embodiments, the number of the cell injection ports 10 may be the same as that of the cell culturing rooms 60, and the cell injection ports 10 may be used for injecting cells different from each other into the cell culturing rooms 60. The cell culturing rooms 60 may be connected, in parallel or in series, to the reagent injection port 30 by at least one reagent injection channel 70. The reagent may be a cell culturing solution or a stimulating material.

Although not shown, the apparatus for culturing and analyzing cells may include a body, which may be formed of silicon, glass, plastic polymer, or any combination thereof. The cell injection channel 80 and the reagent injection channel 70 may be fluid channels provided in the body of the apparatus to have a depth ranging from 30 μm to 50 μm, and the cell injection port 10 and the reagent injection port 30 may be holes formed in the body of the apparatus and connected to the cell injection channel 80 and the reagent injection channel 70, respectively.

A cell-containing solution to be injected into the cell injection ports 10 may contain a specific cell, and it may be delivered into at least one of the cell culturing rooms 60 via the cell injection channels 80. Each of the cell culturing rooms 60 may include a cell capturing structure 65 preventing the cell from being separated from the cell culturing room 60. The cell capturing structure 65 may include a plurality of pillars arranged along an edge of the cell culturing room 60, from a plan view. In example embodiments, an interval between the pillars may be about 5 μm.

The cell culturing rooms 60 may be arranged in the form of a matrix. The reagent injection channel 70 may include a plurality of channels branching off from the reagent injection port 30, where ones of the cell culturing rooms 60 in each row of the matrix arrangement may be connected in series to the corresponding one of the channels. The apparatus for culturing and analyzing cells may further include at least one cell-containing solution exhaust channel 90, each of which may be connected in common to ones of the cell culturing rooms 60 in each row of the matrix arrangement, and a cell-containing solution outlet 40 connected to the cell-containing solution exhaust channel 90 to exhaust the cell-containing solution. The cell culturing rooms 60 may be arranged to have the form of a matrix with dimension M by N. In example embodiments, a total area of the cell culturing rooms 60 may be configured in such a way that it can be completely seen by a microscope with 40× magnification. Example embodiments of the inventive concepts may not be limited to the depicted example, in which the cell culturing rooms 60 are arranged to have the form of 4×8 matrix.

A cell-containing solution and/or a buffer solution, which may be used to inject the specific cell into the cell culturing room 60, may be exhausted through the cell-containing solution outlet 40. In example embodiments, the cell culturing rooms 60 may be configured to include different cells from each other, thereby forming a multiple cell array. A plurality of injection steps may be performed to the multiple cell array.

When the steps of injecting the cells into the cell culturing rooms 60 are finished, a cell culturing solution may be injected through the reagent injection port 30. Thereafter, a culturing process may be performed on the apparatus, in which the cell culturing solution has been injected, for one or two days by using a cell culturing device. Next, processes of inspecting the cells and analyzing the cell reaction patterns caused by the stimulating materials may be further performed.

A stimulating material may be injected into all of the cell culturing rooms 60 through the reagent injection port 30. Cell reactions of the cells caused by the stimulating material may be analyzed by examining visualized patterns occurring in the cell culturing rooms 60. For example, the cell reaction patterns may seem like the examples of FIG. 1.

The apparatus for culturing and analyzing cells may include the reagent outlet 50, which may be connected to portions of the cell culturing rooms 60 opposite the reagent injection port 30 to exhaust the reagent. After the cell culturing process, the culturing solution and/or the buffer solution may be exhausted through the reagent outlet 50.

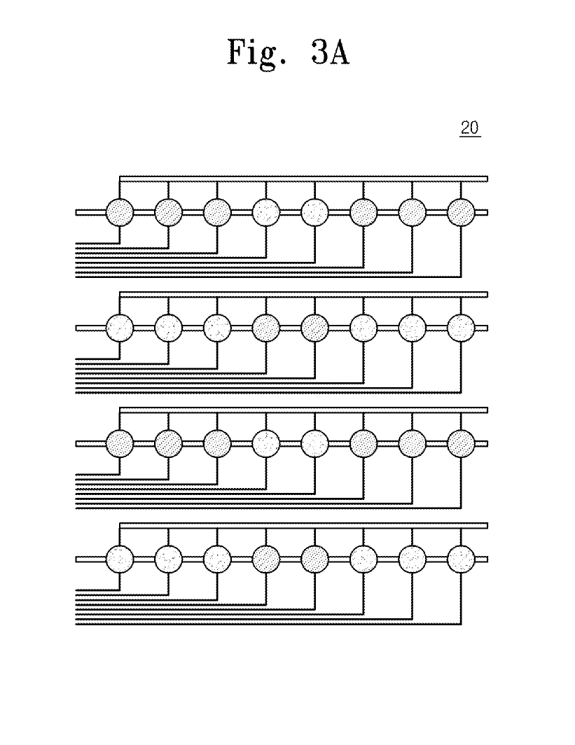

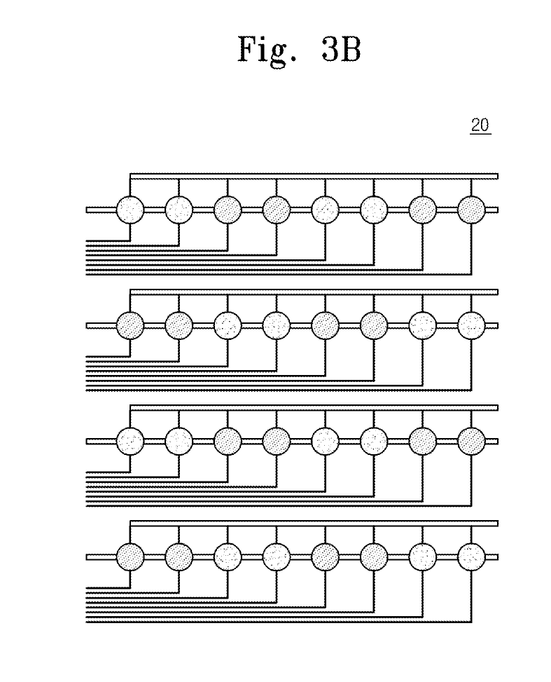

FIGS. 3A and 3B are plan views illustrating results of cell culturing obtained by using an apparatus for culturing a cell and analyzing a cell reaction according to example embodiments of the inventive concept, and in particular, obtained from two different cells arranged at desired culturing rooms.

Referring to FIGS. 3A and 3B, the dark shaded patterns represent some of the cell culturing rooms, in which green fluorescent protein gene was injected to realize green fluorescence, and the light shaded patterns represent others of the cell culturing rooms, in which the fluorescent protein gene was not injected.

Two different cells were injected into predetermined cell culturing rooms, respectively, cultured by a cell culturing process, and then, analyzed through fluorescent images. From the results, it could be found that the two different cells had held properly in the predetermined cell culturing rooms, respectively.

According to example embodiments of the inventive concept, the apparatus for culturing and analyzing cells may be configured in such a way that a plurality of different cells are injected into and cultured at predetermined cell culturing rooms. As a result, reactions of different cells caused by a specific stimulating material can be measured in a visual manner. Accordingly, it is possible to realize an apparatus for culturing and analyzing cells, which can analyze visually not only one-to-one reaction of a cell but also one-to-multi reactions of different cell, caused by a specific stimulating material.

Furthermore, by using the apparatus according to example embodiments of the inventive concept, it is possible to measure simultaneously reactions of different cells caused by a specific stimulating material in a visual manner. Accordingly, it is possible to realize a cell-reaction analyzing method, which can analyze visually not only one-to-one reaction of a cell but also one-to-multi reactions of different cell, caused by a specific stimulating material.

While example embodiments of the inventive concepts have been particularly shown and described, it will be understood by one of ordinary skill in the art that variations in form and detail may be made therein without departing from the spirit and scope of the attached claims.

What is claimed is:

1. An apparatus for culturing and analyzing cells, comprising:
a plurality of cell culturing rooms;
cell injection ports connected to the cell culturing rooms through cell injection channels, respectively, and used for injecting different cells from each other into the cell culturing rooms, the number of the cell injection ports being the same as that of the cell culturing rooms; and
a reagent injection port to inject a reagent into the cell culturing rooms.

2. The apparatus of claim 1, wherein the cell culturing rooms are connected, in parallel or in series, to the reagent injection port by at least one reagent injection channel.

3. The apparatus of claim 1, wherein the cell culturing rooms are arranged in the form of a matrix.

4. The apparatus of claim 3, wherein the reagent injection channel comprises a plurality of channels branching off from the reagent injection port, and
wherein ones of the cell culturing rooms in each row of the matrix arrangement are connected in series to the corresponding one of the channels.

5. The apparatus of claim 4, further comprising:
at least one cell-containing solution exhaust channel, each of which is connected in common to ones of the cell culturing rooms in each row of the matrix arrangement.

6. The apparatus of claim 5, further comprising:
a cell-containing solution outlet connected to the cell-containing solution exhaust channel to exhaust the cell-containing solution.

7. The apparatus of claim 4, wherein the cell culturing rooms are arranged to have the form of an M×N matrix.

8. The apparatus of claim 5, wherein a total area of the cell culturing rooms are configured in such a way that the total area can be completely seen by a microscope with 40× magnification.

9. The apparatus of claim 1, wherein the reagent includes a cell culturing solution or a stimulating material.

10. The apparatus of claim 1, wherein each of the cell culturing rooms comprises a cell capturing structure with a plurality of pillars arranged along an edge of the cell culturing room, from a plan view.

11. The apparatus of claim 1, further comprising:
a reagent outlet connected to portions of the cell culturing rooms opposite the reagent injection port to exhaust the reagent.

12. A method of analyzing a cell reaction, comprising:
injecting different cells from each other into a plurality of cell culturing rooms;
injecting a culturing solution into the cell culturing rooms to culture the cells; and
injecting a stimulating material into all of the cell culturing rooms,
wherein cell reactions of the cells caused by the stimulating material are analyzed by examining visualized patterns occurring in the cell culturing rooms.

13. The method of claim 12, wherein the cell culturing rooms are arranged to have the form of an M×N matrix.

14. The method of claim 13, wherein a total area of the cell culturing rooms are configured in such a way that the total area can be completely seen by a microscope with 40× magnification.
